(19) United States
(12) Patent Application Publication
(10) Pub. No.: US 2023/0101907 A1
(43) Pub. Date: Mar. 30, 2023

(54) STRESS RELEASE DEGREE CALCULATION APPARATUS, STRESS RELEASE DEGREE CALCULATIONMETHOD, AND COMPUTER READABLE RECORDING MEDIUM

(71) Applicant: NEC Corporation, Minato-ku, Tokyo (JP)

(72) Inventors: Terumi UMEMATSU, Tokyo (JP); Masanori TSUJIKAWA, Tokyo (JP); Tasuku KITADE, Tokyo (JP); Yoshiki NAKASHIMA, Tokyo (JP); Jingwen LU, Tokyo (JP)

(73) Assignee: NEC Corporation, Minato-ku, Tokyo (JP)

(21) Appl. No.: 17/911,459

(22) PCT Filed: Mar. 31, 2020
(86) PCT No.: PCT/JP2020/014794

§ 371 Date: Sep. 14, 2022

(51) Int. Cl. A61B 5/16; A61B 5/0205; A61B 5/00

(52) U.S. Cl. CPC A61B 5/165; A61B 5/0205; A61B 5/486; A61B 5/7275

(57) ABSTRACT

The apparatus includes the short-term stress calculation unit calculating a short-term stress value that indicates stress felt by a subject in a first period, the long-term stress calculation unit calculating a long-term stress value that indicates stress felt by the subject in a second period that is longer than the first period and includes the first period, the short-term stress change amount calculation unit calculating a short-term stress change amount of the subject based on the difference between the short-term stress values calculated at different timings, the long-term stress change amount calculation unit calculating a long-term stress change amount of the subject based on the difference between the long-term stress values calculated at different timings, and the stress release degree calculation unit calculating a stress release degree indicating the extent of stress released by the subject based on the short-term stress change amount and the long-term stress change amount.

### TECHNICAL FIELD

The present invention relates to a stress release degree calculation apparatus, a stress release degree calculation method, and a computer readable recording medium.

### BACKGROUND ART

In recent years, an issue in which the sympathetic nerve system is excited due to excessive stress, and the mind and body become fatigued, thus resulting in one's health suffering is of concern. Therefore, it is desired that one manages their stress state, and that stress is regularly relieved. In Patent Document 1, a karaoke system for supporting stress relief by singing is disclosed. In the technique disclosed in Patent Document 1, the stress value of a karaoke user is detected from the heart rate of the user, and the degree to which stress is relieved through singing is calculated.

### LIST OF RELATED ART DOCUMENTS

### Patent Document

Patent Document 1: Japanese Patent Laid-Open Publication No. 2015-172701A

### SUMMARY OF INVENTION

### Problems to be Solved by the Invention

In the technique disclosed in Patent Document 1, stress values are detected when a user performs a login operation and when the user performs a logout operation, and the difference between the two stress values is calculated as a stress relief degree. In this case, the stress relief degree during the period between the two timings can be grasped. However, in daily life, the timing at which stress is released is unknown. There fore, with the invention disclosed in Patent Document 1, the stress release degree in daily life may not be accurately calculated.

An example object of the invention is to provide a stress release degree calculation apparatus, a stress release degree calculation method, and a computer readable recording medium, with which a stress release degree in daily life can be accurately calculated.

### Means for Solving the Problems

In order to achieve the above-described object, a stress management apparatus according to an example aspect of the invention comprising:

a short-term stress calculation unit that calculates a short-term stress value that indicates stress felt by a subject in a first period;

a long-term stress calculation unit that calculates a long-term stress value that indicates stress felt by the subject in a second period that is longer than the first period and includes the first period;

a short-term stress change amount calculation unit that calculates a short-term stress change amount of the subject based on the difference between the short-term stress values calculated at different timings;

a long-term stress change amount calculation unit that calculates a long-term stress change amount of the subject based on the difference between the long-term stress values calculated at different timings; and

a stress release degree calculation unit that calculates a stress release degree indicating the extent of stress released by the subject based on the short-term stress change amount and the long-term stress change amount.

In order to achieve the above-described object, a stress management method according to an example aspect of the invention comprising:

a step of calculating a short-term stress value that indicates stress felt by a subject in a first period;

a step of calculating a long-term stress value that indicates stress felt by the subject in a second period that is longer than the first period and includes the first period;

a step of calculating a short-term stress change amount of the subject based on the difference between the short-term stress values calculated at different timings;

a step of calculating a long-term stress change amount of the subject based on the difference between the long-term stress values calculated at different timings; and

a step of calculating a stress release degree indicating the extent of stress released by the subject based on the short-term stress change amount and the long-term stress change amount.

In order to achieve the above-described object, a computer-readable recording medium according to an example aspect of the invention is a computer-readable recording medium that includes a program including instructions recorded thereon, the instructions causing a computer to carry out:

a step of calculating a short-term stress value that indicates stress felt by a subject in a first period;

a step of calculating a long-term stress value that indicates stress felt by the subject in a second period that is longer than the first period and includes the first period;

a step of calculating a short-term stress change amount of the subject based on the difference between the short-term stress values calculated at different timings;

a step of calculating a long-term stress change amount of the subject based on the difference between the long-term stress values calculated at different timings; and

a step of calculating a stress release degree indicating the extent of stress released by the subject based on the short-term stress change amount and the long-term stress change amount.

### Advantageous Effects of the Invention

According to the invention, stress management in daily life can be accurately calculated.

### BRIEF DESCRIPTION OF THE DRAWINGS

FIG. 1 is a diagram illustrating the short-term stress and long-term stress on a time axis.

FIG. 2 is a diagram conceptually illustrating the short-term stress and long-term stress.

FIG. 3 is a block diagram illustrating a schematic configuration of a stress release degree calculation apparatus.

FIG. 4 is a diagram illustrating a specific configuration of the stress release degree calculation apparatus.

FIG. 5 is a diagram illustrating a relationship between the stress release degree and the short-term stress change amount and the long-term stress change amount.

FIG. 6 is a diagram for describing the method for calculating the short-term stress tolerable amount.

FIG. 7 is a diagram illustrating an example of a correspondence table that is referred to when the stress degree is calculated.

FIG. 8 is a diagram illustrating an exemplary display screen when a notification regarding the stress release degree is made.

FIG. 9 is a diagram illustrating an exemplary message according to the stress release degree.

FIG. 10 is a diagram illustrating an exemplary display screen when a notification regarding the stress tolerable remaining amount is made.

FIG. 11 is a diagram illustrating an exemplary display screen of a stress relieving method according to the stress degree.

FIG. 12 is a diagram illustrating an exemplary display screen when a notification regarding various types of information regarding stress is made.

FIG. 13 is a flow diagram illustrating operations when a notification regarding the long-term stress value and short-term stress value are calculated and the management information is made.

FIG. 14 is a flow diagram illustrating operations when calculating the short-term stress value, the short-term stress change amount, and the short-term stress tolerable remaining amount.

FIG. 15 is a flow diagram illustrating operations when calculating the long-term stress value, the long-term stress change amount, and the long-term stress tolerable remaining amount.

FIG. 16 is a flow diagram illustrating operations when calculating the stress tolerable amount.

FIG. 17 is a flow diagram illustrating operations when calculating the stress release degree.

FIG. 18 is a flow diagram illustrating operations when calculating the stress degree.

FIG. 19 is a block diagram showing an example of a computer that realizes the stress release degree calculation apparatus.

### EXAMPLE EMBODIMENT

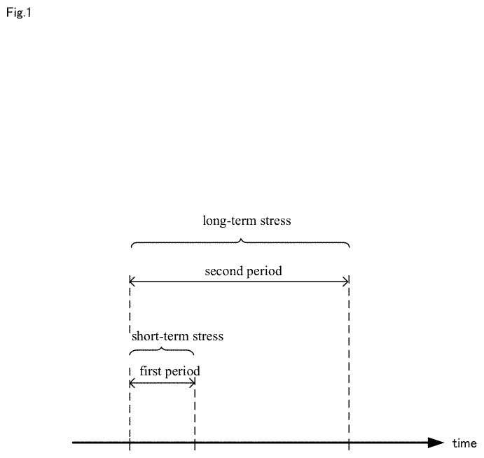

Hereinafter, the configuration of a stress management apparatus in an example embodiment of the present invention will be described with reference to FIGS. 1 to 19. A stress management apparatus 15 is an apparatus that manages stress felt by an object, and in the following, the object is referred to as a subject (subject to be measured). The stress management apparatus can calculate short-term stress, which is a short-term stress stimulus, and long-term stress, which is a long-term stress stimulus, that are felt by the subject.

### Description of Definitions

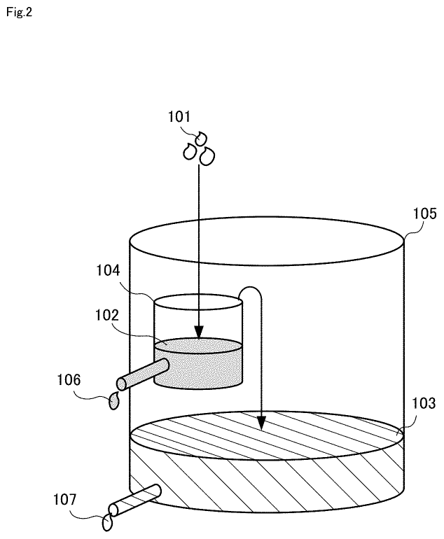

First, the definitions of the short-term stress and long-term stress will be described using FIGS. 1 and 2.

FIG. 1 is a diagram illustrating the short-term stress and long-term stress on a time axis. The short-term stress is stress felt by the subject in a first period. The first period is a period of several hours or one day, for example. The long-term stress is stress felt by the subject in a second period. The second period is a period that is longer than the first period and includes the first period, and is a period of several days, several weeks, or several months, for example. That the second period includes the first period means that, when the first period is a period from timing A to timing B, for example, the second period is at least a period from timing A or before to timing B or after.

FIG. 2 is a diagram conceptually illustrating the short-term stress and long-term stress. The mechanism of the subject accumulating stress will be conceptually described using FIG. 2. In this description, it is assumed that the subject has a short-term stress container 104 for storing a short-term stress 102 and a long-term stress container 105 for storing a long-term stress 103.

When the subject feels a stress stimulus (hereinafter, referred to as “acute stress”) 101 that lasts several seconds or several minutes, for example, the acute stress 101 is first stored in the short-term stress container 104. That is, the subject feels the short-term stress 102 by successively storing the acute stress 101 in the short-term stress container 104.

The amount of short-term stress 102 that the short-term stress container 104 can store is an amount of short-term stress 102 that the subject can tolerate, and is referred to as a “short-term stress tolerable amount”. If the subject successively stores the short-term stress 102, the amount of short-term stress 102 exceeds the short-term stress tolerable amount. Then, the short-term stress 102 spills out from the short-term stress container 104. The short-term stress 102 that has spilled out is stored in the long-term stress container 105. That is, when the subject can no longer store the short-term stress 102 in the short-term stress container 104, the subject feels the long-term stress 103.

Note that the remaining amount of short-term stress 102 before the short-term stress tolerable amount is reached is referred to as a “short-term stress tolerable remaining amount”. Also, when the subject performs coping (stress handling), a stress 106 that is released by coping is discharged from the short-term stress container 104.

The amount of long-term stress 103 that the long-term stress container 105 can store is an amount of long-term stress 103 that the subject can tolerate, and is referred to as a “long-term stress tolerable amount”. If the subject successively stores the long-term stress 103, the amount of long-term stress 103 exceeds the long-term stress tolerable amount. Then, the long-term stress 103 spills out from the long-term stress container 105. In this case, the subject may possibly suffer from a symptom detrimental to daily life, such as a psychiatric disorder.

Note that the remaining amount of long-term stress 103 before the long-term stress tolerable amount is reached is referred to as a “long-term stress tolerable remaining amount”. Also, when the subject performs coping, for example, a stress 107 in an amount corresponding to the released amount by coping is discharged from the long-term stress container 105.

Next, the stress management apparatus according to the present example embodiment will be described.

[Description of Configuration]

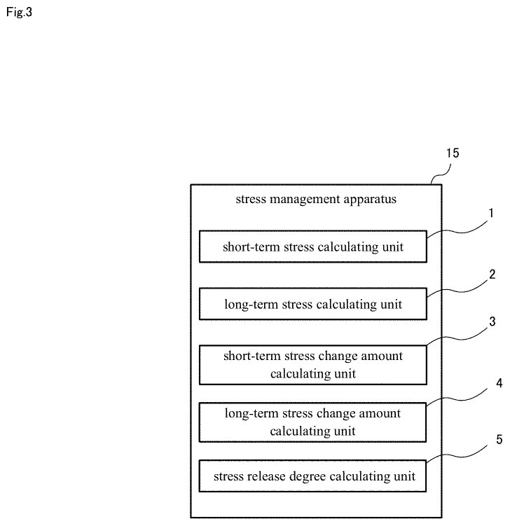

FIG. 3 is a block diagram illustrating a schematic configuration of a stress release degree calculation apparatus 15.

The stress release degree calculation apparatus 15 includes a short-term stress calculating unit 1, a long-term stress calculating unit 2, a short-term stress change amount calculating unit 3, a long-term stress change amount calculating unit 4, and a stress release degree calculating unit 5.

The short-term stress calculating unit 1 calculates a short-term stress value, which indicates the amount of stress felt by a subject in a first period. When a conceptual description is given using FIG. 2, the short-term stress value is an amount of the short-term stress 102 accumulated in the short-term stress container 104 in the first period.

The long-term stress calculating unit 2 calculates a long-term stress value, which indicates the amount of stress felt by the subject in a second period that is longer than the first period and includes the first period. When a conceptual description is given using FIG. 2, the long-term stress value is an amount of the long-term stress 103 accumulated in the long-term stress container 105 in the second period.

The short-term stress change amount calculating unit 3 calculates a short-term stress change amount of the subject based on the difference between short-term stress values that are calculated at different timings.

The long-term stress change amount calculating unit 4 calculates a long-term stress change amount of the subject based on the difference between long-term stress values that are calculated at different timings.

The stress release degree calculating unit 5 calculates a stress release degree that indicates the extent of stress released by the subject based on the short-term stress change amount and the long-term stress change amount.

The stress release degree calculation apparatus 15 can accurately calculate the stress release degree in daily life. The subject can recognize one's own stress situation from the calculated stress release degree.

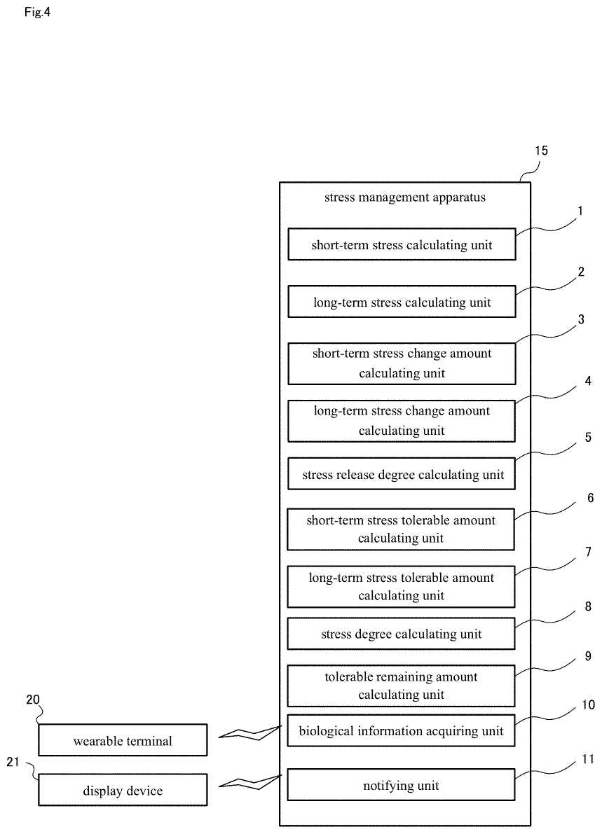

Next, the configuration and functions of the stress release degree calculation apparatus 15 in the present example embodiment will be described in detail using FIGS. 4 to 18.

FIG. 4 is a block diagram illustrating a specific configuration of the stress release degree calculation apparatus 15.

The stress release degree calculation apparatus 15 includes a short-term stress tolerable amount calculating unit 6, a long-term stress tolerable amount calculating unit 7, a stress degree calculating unit 8, an tolerable remaining amount calculating unit 9, a biological information acquiring unit 10, and a notifying unit 11, in addition to the short-term stress calculating unit 1, the long-term stress calculating unit 2, the short-term stress change amount calculating unit 3, the long-term stress change amount calculating unit 4, and the stress release degree calculating unit 5.

The biological information acquiring unit 10 acquires biological information regarding a subject. Specific examples of the biological information include information regarding a perspiration amount, cutaneous temperature, body motion, a heart rate, an electrocardiogram, a pulse wave, a pulse, blood pressure, respiration, pupils, a brain wave, and myoelectric or gastric electrical potential of the subject. The biological information may be one piece of information from among these pieces of information, or may also be information in which two or more pieces of information are combined.

The biological information is detected by a wearable terminal 20 attached to a subject. The wearable terminal 20 includes a sensor for detecting a biological signal of the subject. A specific example of the sensor includes, when the biological information is a heart rate, electrodes for measuring an electrocardiogram or an optical sensor for optically detecting the movement of blood. Upon acquiring a biological signal output from the sensor, the wearable terminal 20 calculates biological information such as a heart rate, cutaneous temperature, an electrodermal response, and acceleration using the biological signal, and transmits the calculated biological information to the stress release degree calculation apparatus 15. The biological information acquiring unit 10 acquires the biological information transmitted from the wearable terminal 20. Note that the biological information acquiring unit 10 may also be configured to receive a biological signal from the wearable terminal 20 and calculate biological information from the received biological signal.

The short-term stress calculating unit 1 calculates a short-term stress value using machine learning from the biological information acquired by the biological information acquiring unit 10. Specific examples of the calculation method include methods described in “T. Umematsu, A. Sano, S. Taylor, R. Picard, “Improving Students' Daily Life Stress Forecasting using LSTM Neural Networks.” P. 1-4., 2019 IEEE EMBS International Conference on Biomedical & Health Informatics (BHI), 2019.” And “S. A. Taylor et al., “Personalized Multitask Learning for Predicting Tomorrow's Mood, Stress, and Health,” IEEE Transactions on Affective Computing, no. 99, pp. 1-14, 2017”.

The long-term stress calculating unit 2 calculates a long-term stress value from the biological information or a questionnaire asked in advance. An example of the method for calculating the long-term stress value from the biological information includes a method that is the same as the method for calculating the short-term stress value. Also, other specific examples of the method for calculating the long-term stress value from the biological information include methods described in “A. Sano, “Measuring College Students” Sleep, Stress, Mental Health and Wellbeing with Wearable Sensors and Mobile Phones”, Massachusetts Institute of Technology, 2015.”, and “Y. Nakashima et al., “An Effectiveness Comparison between the Use of Activity State Data and That of Activity Magnitude Data in Chronic Stress Recognition,” ACII workshop, 2019”. Also, a specific example of the questionnaire includes Perceived Stress Scale (PSS) questionnaire (URL:http://www.shinyo.pro/blog/upload-images/20165218835.jpg). The results of the questionnaire are input to the stress release degree calculation apparatus 15 at a timing at which the stress release degree calculation apparatus 15 starts stress management, for example.

When the long-term stress value is calculated from a PSS questionnaire, the long-term stress calculating unit 2 calculates a score of the PSS questionnaire, and calculates the long-term stress value from the score. A specific example of the calculation method for the score of a PSS questionnaire includes a method described in “S. Cohen, R. C. Kessler, and L. U. Gordon, “Measuring Stress: A Guide for Health and Social Scientists,” Oxford University Press, 1997.”.

The short-term stress change amount calculating unit 3 calculates a short-term stress change amount of the subject based on the difference between short-term stress values calculated at different timings. Specifically, if the change amount of the short-term stress value is a decreasing amount, the short-term stress change amount calculating unit 3 calculates the short-term stress change amount as a short-term stress release amount, which is the amount of the short-term stress released by the subject. Also, if the change amount of the short-term stress value is an increasing amount, the short-term stress change amount calculating unit 3 calculates the short-term stress change amount as a short-term stress accumulation amount, which is the amount of the short-term stress accumulated by the subject.

The long-term stress change amount calculating unit 4 calculates a long-term stress change amount of the subject based on the difference between long-term stress values calculated at different timings. Specifically, if the change amount of the long-term stress value is a decreasing amount, the long-term stress change amount calculating unit 4 calculates the long-term stress change amount as a long-term stress release amount, which is an amount of the long-term stress released by the subject. Also, if the change amount of the long-term stress value is an increasing amount, the long-term stress change amount calculating unit 4 calculates the long-term stress change amount as a long-term stress accumulation amount, which is an amount of the long-term stress accumulated by the subject.

The stress release degree calculating unit 5 calculates a stress release degree, which indicates an extent of stress released by the subject, based on the short-term stress change amount calculated by the short-term stress change amount calculating unit 3 and the long-term stress change amount calculated by the long-term stress change amount calculating unit 4.

In the following, the short-term stress change amount, the long-term stress change amount, and the stress release degree will be described.

In the present example embodiment, the stress release degree is represented by nine levels from 1 to 9, and the larger the value is, the larger amount of stress is released. The stress release degree is calculated based on the relationship between a long-term stress change amount Δl and a short-term stress change amount Δs.

The long-term stress change amount Δl can be obtained from a long-term stress value L_{T} at a given timing T and a long-term stress value L_{T+1} at a next timing T+1 adjacent to the timing T using the following formula.

Δl=L_{T+1}−L_{T}

When the long-term stress change amount Δl decreases (when Δl<0), the long-term stress is released, and the long-term stress change amount Δl indicates a released amount of the long-term stress. The released amount of the long-term stress is the released amount the subject released the long-term stress. When the long-term stress change amount Δl increases (when Δl>0), the long-term stress is accumulated, and the long-term stress change amount Δl indicates the accumulated amount of the long-term stress. The accumulated amount of the long-term stress is the accumulated amount the subject accumulated the long-term stress.

The short-term stress change amount Δs can be obtained from a short-term stress value S_{T} at a given timing T and a short-term stress value S_{T+1} at a next timing T+1 adjacent to the timing T using the following formula.

Δs=S_{T+1}−S_{T}

When the short-term stress change amount Δs decreases (when Δs<0), the short-term stress is released, and the short-term stress change amount Δs indicates the released amount of the short-term stress. When the short-term stress change amount Δs increases (when Δs>0), the short-term stress is accumulated, and the short-term stress change amount Δs indicates the accumulated amount. Note that A s may also be calculated using data at a timing T+N (N is any number) that is not adjacent to the timing T. Here, A s=S_{T+N}−S_{T} is Δs in a period shorter than the second period for which the long-term stress is calculated.

When the long-term stress change amount Δl and the short-term stress change amount Δs are released amounts and the larger the released amounts Δl and Δs are, the higher the stress release degree is. Also, when the long-term stress change amount Δl and the short-term stress change amount Δs are accumulated amounts and the larger the accumulated amounts Δl and Δs are, the lower the stress release degree is.

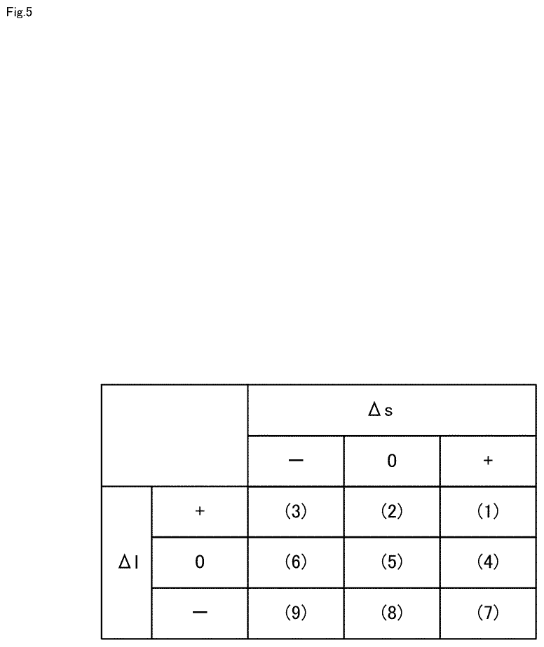

FIG. 5 is a diagram illustrating a relationship between the stress release degree and the short-term stress change amount Δs and the long-term stress change amount Δl. As described above, the stress release degree is represented by nine levels from (1) to (9). (1) in FIG. 5 indicates that the stress release degree is the lowest, and (9) in FIG. 5 indicates that the stress release degree is the highest.

As described in FIG. 2, when the long-term stress exceeds the long-term stress tolerable amount, the subject may possibly suffer from a condition detrimental to daily life such as a psychiatric disorder. That is, the stress release degree of the subject is increased in response to the long-term stress being released rather than the short-term stress being released.

Note that FIG. 5 shows an example of the stress release degree calculated by the stress release degree calculating unit 6, and the stress release degree is not limited thereto. The stress release degree may be represented by nine or more levels, or eight or smaller levels.

As a result of calculating the stress release degree, the subject can easily find a stress release method suitable for him/herself, and can easily manage the mental health. Also, the subject can recognize whether the short-term stress is released or the long-term stress is released, and therefore the subject can easily find an effective stress release method.

Return to FIG. 4. The short-term stress tolerable amount calculating unit 7 calculates a short-term stress tolerable amount indicating a short-term stress amount that can be tolerated by the subject based on the short-term stress value and the long-term stress value.

The method for calculating the short-term stress tolerable amount by the short-term stress tolerable amount calculating unit 7 will be described. The short-term stress tolerable amount calculating unit 7 calculates a largest value among short-term stress values that the short-term stress calculating unit 1 calculated before the long-term stress value has increased, as a short-term stress tolerable amount.

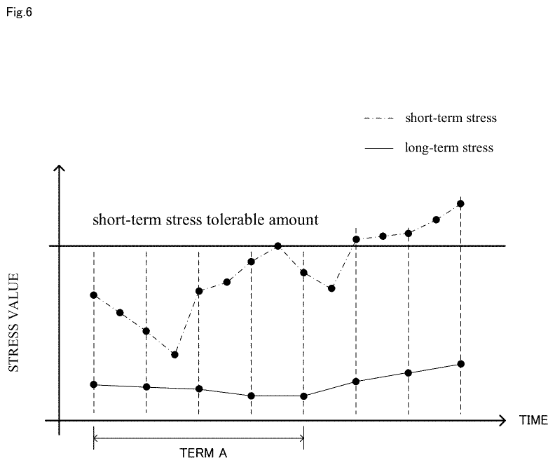

FIG. 6 is a diagram for describing the method for calculating the short-term stress tolerable amount. The horizontal axis of the graph shown in FIG. 6 shows time, and the vertical axis shows a stress value. The solid polygonal line in FIG. 6 shows transitions of the long-term stress value, and the one dot chain polygonal line shows transitions of the short-term stress value.

As described in FIG. 2, when the short-term stress value exceeds the short-term stress tolerable amount, the long-term stress value increases. Therefore, the short-term stress tolerable amount calculating unit 7 calculates the short-term stress tolerable amount based on the timing at which the long-term stress value increases. For example, the short-term stress tolerable amount calculating unit 7 calculates the largest value (solid straight line in FIG. 6) of the short-term stress value in a period (period A in FIG. 6) in which the increased amount from a long-term stress value calculated at a given timing to a long-term stress value calculated immediately thereafter is a predetermined amount or less, as the short-term stress tolerable amount.

Note that the method for determining the period A in FIG. 6 is not limited thereto. For example, the period until the long-term stress value that was decreasing or transitioning without change increases may be determined as the period A in FIG. 6. Also, the largest value of the short-term stress value in the period A is determined as the short-term stress tolerable amount, but there is no limitation thereto. For example, an integrated value, an average value, or a median value of the short-term stress value in the period A may also be determined as the short-term stress tolerable amount.

Also, if the long-term stress value has not been obtained in a given period, the largest value, an integrated value, an average value, or a median value of the short-term stress value in a period of a length similar to that when the long-term stress value is to be calculated may also be calculated as the short-term stress tolerable amount.

As a result of calculating the short-term stress tolerable amount using such a calculation method, the subject can recognize the stress tolerable amount in accordance with the subject's current situation. That is, the subject can deduce that the extent of influence of stress felt from the same event on the mind and body differs depending on the situation, and can easily prevent overstress.

Return to FIG. 4. The long-term stress tolerable amount calculating unit 7 calculates the long-term stress tolerable amount indicating the long-term amount value that the subject can tolerate based on the long-term stress value when it is determined that there is no problem in the health condition of the subject.

The method for calculating the long-term stress tolerable amount by the long-term stress tolerable amount calculating unit 7 will be described. The long-term stress tolerable amount calculating unit 7 detects long-term stress values that can be determined to indicate a healthy state, and calculates the largest value, average value, or median value thereof as the long-term stress tolerable amount, for example. This long-term stress value may be calculated from biological information, or may also be calculated from a score of a PSS questionnaire. The healthy state is a state in which there is no problem with the mind and body of the subject, which is determined from a past record such as a service record, a medical check-up record, or a medical care record, for example. Also, the service record refers to a state in which the subject consistently works regular working hours without coming late to work by a large amount. The medical check-up record refers to a state in which the health state of the subject has not worsened relative to the previous record, or a state in which the BMI is a normal value or less. The medical care record refers to a state in which there is no hospital visit record, or the frequency of hospital visits has decreased. In addition, the long-term stress tolerable amount may also be a long-term stress value immediately before hospitalization, or a long-term stress value immediately before one's health suffers. The long-term stress tolerable amount may also be the largest value of the long-term stress value in a given period before then, instead of the value immediately before.

The tolerable remaining amount calculating unit 9 calculates the long-term stress tolerable remaining amount and the short-term stress tolerable remaining amount that have been described above using FIG. 2. Specifically, the tolerable remaining amount calculating unit 9 calculates the remaining amount of the short-term stress before the short-term stress tolerable amount is reached, that is, the difference between the short-term stress value at the current point in time and the short-term stress tolerable amount, as the short-term stress tolerable remaining amount. Also, the tolerable remaining amount calculating unit 9 calculates the remaining amount of the long-term stress before the long-term stress tolerable amount is reached, that is, the difference between the long-term stress value at the current point in time and the long-term stress tolerable amount, as the long-term stress tolerable remaining amount.

Note that the tolerable remaining amount calculating unit 9 calculates, if the stress value exceeds the stress tolerable amount, the difference between the stress value and the stress tolerable amount, as a stress excess amount. Specifically, if the short-term stress value exceeds the short-term stress tolerable amount, the tolerable remaining amount calculating unit 9 calculates the difference between the short-term stress value and the short-term stress tolerable amount, as a short-term stress excess amount. Also, if the long-term stress value exceeds the long-term stress tolerable amount, the tolerable remaining amount calculating unit 9 calculates the difference between the long-term stress value and the long-term stress tolerable amount, as a long-term stress excess amount.

As a result of calculating the stress tolerable amount or the stress tolerable remaining amount, the subject can recognize how much stress can be tolerated. As a result, the subject can manage own schedule appropriately while managing own mental health.

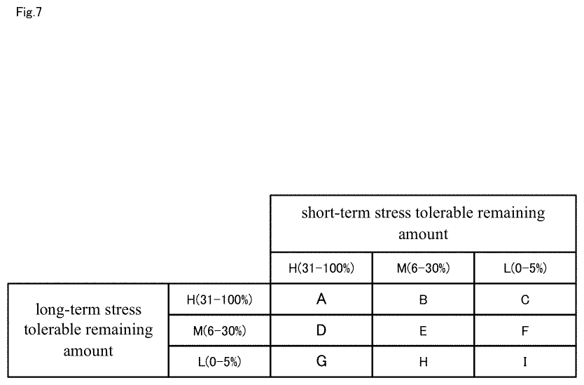

The stress degree calculating unit 8 calculates a stress degree that indicates the extent of stress felt by the subject based on a short-term stress remaining amount before the short-term stress tolerable amount is reached and a long-term stress remaining amount before the long-term stress tolerable amount is reached. Specifically, after the tolerable remaining amount calculating unit 9 has calculated the short-term stress tolerable remaining amount and the long-term stress tolerable remaining amount, the stress degree calculating unit 8 calculates the stress degree of the stress felt by the subject from the correspondence table shown in FIG. 7, at a timing at which the long-term stress calculating unit 2 calculates a long-term stress value, for example.

FIG. 7 is a diagram illustrating an example of a correspondence table that is referred to when the stress degree is calculated. FIG. 7 shows (a) correspondence (relationship) between the stress degree and the long-term stress tolerable remaining amount and short-term stress tolerable remaining amount. The stress tolerable remaining amount increases in the order of L (0-5%), M (6-30%), and H (31-100%), where H (31-100%) indicates that the stress tolerable remaining amount is 31 to 100% of the stress tolerable amount, for example. Note that, in FIG. 7, “%” is used as an exemplary unit of the stress tolerable remaining amount.

The stress degree calculating unit 9 calculates the stress degree in nine levels from A to I. “A” indicates that the stress degree is the lowest, and “I” indicates that the stress degree is the highest. For example, when the short-term stress tolerable remaining amount and the long-term stress tolerable remaining amount are each “H (31-100%)”, which indicates a large amount, the stress degree calculating unit 9 calculates “A”, which indicates that the stress degree is low.

The notifying unit 11 displays management information based on the long-term stress value and short-term stress value in a display device 21, for example. The display device 21 is a PC, a smartphone, or a tablet terminal, for example. Also, the display device 21 may also be a display unit of the wearable terminal 20, and the notifying unit 11 may notify the wearable terminal 20 of information.

The notifying unit 11 displays at least one of the stress release degree and information based on the stress release degree in the display device 21, as the management information.

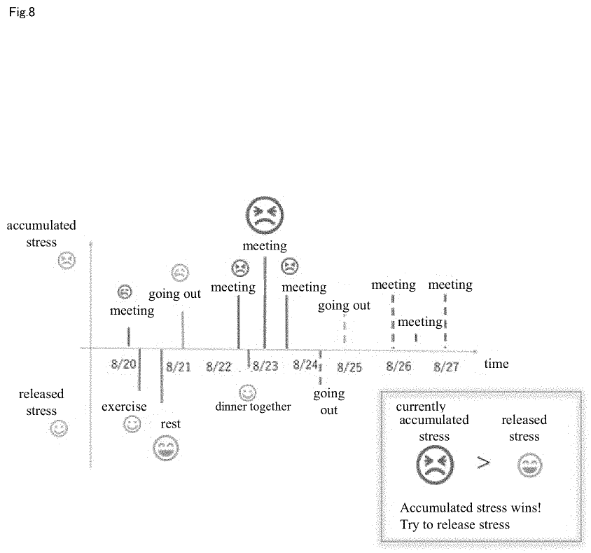

FIG. 8 is a diagram illustrating an exemplary display screen when a notification regarding the stress release degree is made. In FIG. 8, the stress release degree is displayed using face marks.

In this diagram, whether stress is accumulated or released with respect to events is displayed. For example, with respect to an event “meeting” on 8/20, it is displayed that stress is accumulated. Also, the notifying unit 11 displays a message for prompting a user to release stress in accordance with the current stress release degree, such as the message in a square in FIG. 8, as the information based on the stress release degree.

Also, in FIG. 8, a message for proposing a stress releasing method may be displayed in accordance with the stress release degree, such as “From the prior history, the rest taken on 8/20 was particularly useful for releasing stress. When feeling stressed, please release stress similarly, this will be beneficial to your mind and body health.”, for example. Also, when the amount of accumulated stress is large, a message may also be displayed. An example of such a message being “It seems like you felt a lot of stress in the meeting on 8/23. Please take care to deal with stress next time by, for example, refreshing after the meeting, or changing your attitude toward the meeting a little! In general, high quality sleep has a large effect on reducing stress. Please have a good sleep at night after a meeting.”.

Note that, in FIG. 8, event information such as “meeting” and “going out” are displayed in the graph in time series based on the stress release degree, in an associated manner, but the configuration may also be such that only the graph in time series based on the stress release degree is displayed, and the event information is not displayed.

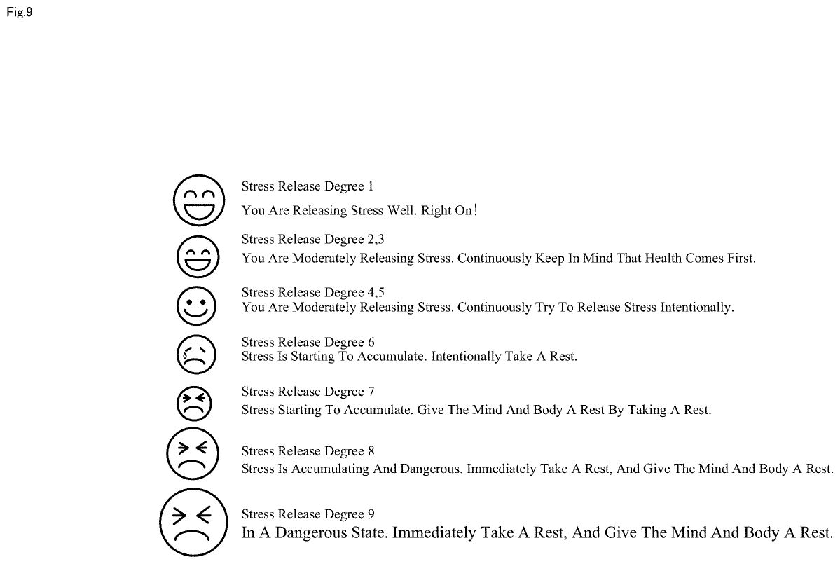

Also, the notifying unit 11 may also display a message according to the stress release degree, as information based on the stress release degree, as shown in FIG. 9.

FIG. 9 is a diagram illustrating an exemplary message according to the stress release degree. The release degrees 1 to 9 in FIG. 9 correspond to (1) to (9) in FIG. 5, and the release degree 1 in FIG. 9 corresponds to (1) in FIG. 5.

Furthermore, the notifying unit 11 may also display, as the management information, at least one of the short-term stress tolerable remaining amount, which is the short-term stress remaining amount before the short-term stress tolerable amount is reached, and the long-term stress tolerable remaining amount, which is the long-term stress remaining amount before the long-term stress tolerable amount is reached, in the display device 21.

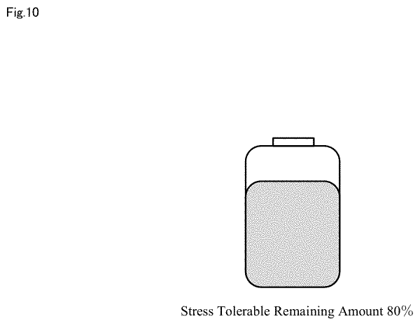

FIG. 10 is a diagram illustrating an exemplary display screen when a notification regarding the stress tolerable remaining amount is made. As shown in FIG. 10, the stress tolerable remaining amount may be displayed by using an image in the fashion of a remaining battery charge amount. The stress tolerable remaining amount in FIG. 10 may be the short-term stress tolerable remaining amount, or may also be the long-term stress tolerable remaining amount. Note that the stress tolerable remaining amount may also be displayed by using an image in which water is accumulated in a water pot.

Also, the notifying unit 11 may display at least one of the stress degree and information based on the stress degree in the display device 21, as the management information. The information based on the stress degree is information for relieving stress felt by the subject, for example.

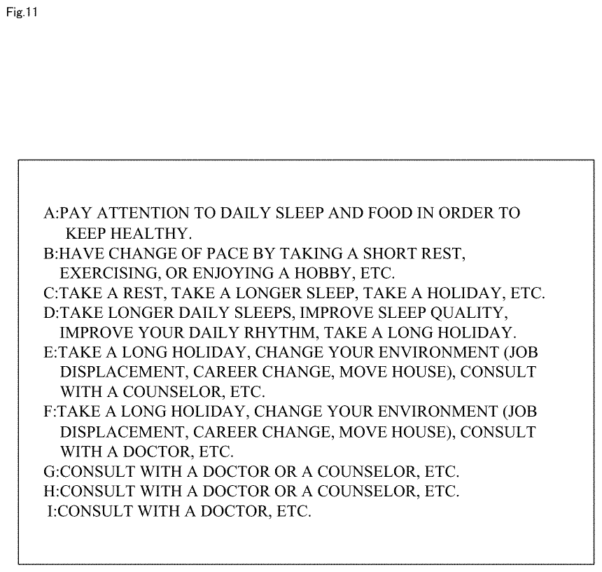

FIG. 11 is a diagram illustrating an exemplary display screen of a stress relieving method according to the stress degree.

A stress coping plan is proposed to the subject by displaying a message in accordance with the stress degrees A to I described in FIG. 7. In this case, the text display color is changed according to the stress degree. For example, when the stress degree is high, red is used as the text display color for prompting attention, for example. Also, when the stress degree is low, in the case of “B”, for example, a message for encouraging the subject may also be displayed such as “Feeling a bit worn out? Stay healthy by resting when you can!”.

Also, the notifying unit 11 displays time series data indicating the short-term stress value and long-term stress value and events regarding the subject in the display device 21, in an associated manner, as the management information.

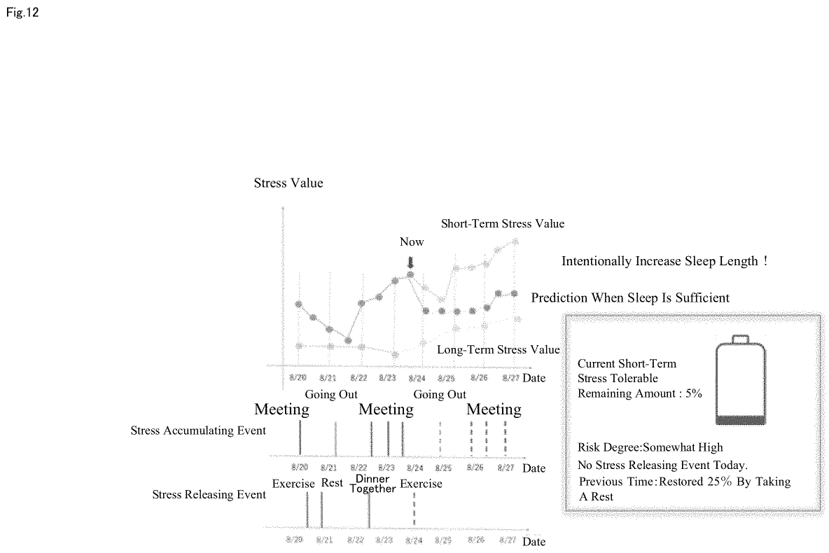

FIG. 12 is a diagram illustrating an exemplary display screen when a notification regarding various types of information regarding stress is made.

In FIG. 12, a stress value time series graph, stress accumulation events, and stress release events are displayed in a linked manner.

The event information is a meeting, going out, or the like, and is extracted from schedule information personally associated with the subject. The schedule information may be automatically extracted, or the subject may manually input and describe events freely. Also, regarding an exercise or rest, whether the subject is exercising or resting is determined by estimating personal motion information from the biological information (acceleration and the like) of the wearable terminal 20.

A specific example of determination is as follows.

(i) a=√(x\hat{ }2+y\hat{ }2+z\hat{ }2) is calculated regarding three axes of accelerations x, y, and z (every 4 Hz, if sampling rate is 4 Hz).
(ii) Regarding a, statistical values such as a total value a_sum and an average value in a given period (e.g., 10 minutes) are calculated.
(iii) A threshold value is determined, if a_sum is a given value or more, “exercise” (=motion amount is large) is determined, and if a_sum is smaller, “rest” (=motionless) is determined.
(iv) Regarding how to determine the threshold value, several persons are asked to actually “exercise” and “rest”, and the threshold value is determined based on the average values of a_sum at these moments.

Note that, as shown in FIG. 12, a tolerable remaining amount exemplary display is displayed on the screen, and the stress degree, a stress handling method, and the like may be displayed, but only one of these? may also be displayed.

Also, in FIG. 12, in addition to the actual values, predicted values from “Now”, which indicates the current time, and thereafter are displayed. The method for calculating the predicted values includes a method in which a model that has been trained using feature amounts regarding stress that are obtained from biological information as inputs, and stress values of tomorrow and thereafter as correct answers, for example. These predicted values may not be displayed.

Also, the method for calculating the predicted values in the case of “prediction when sleep is sufficient”, in FIG. 12, includes a following exemplary method.

When the subject has slept 7 to 8 hours, which is desirable in general, for several days, in the stress release degree levels of nine levels from (1) to (9) described in FIG. 5, a stress value at one level up from the current stress release degree is calculated. For example, when the current stress release degree is (5), if the subject has slept sufficiently, the stress release degree is changed to (6). Then, the stress value when the stress release degree was (6) in the past is displayed. On the other hand, if there is no case where the stress release degree was (6), a stress value that is obtained by decreasing the average stress value for several days by 10% is calculated as the predicted value. Note that 10% is merely an example. If there is no past information, a stress value that is obtained by decreasing the current stress value by 10% is calculated as the predicted value, for example.

Note that, in FIG. 12, event information is displayed in an associated manner, but only time series data indicating the short-term stress value and long-term stress value may also be displayed.

The subject can know his/her own stress situation from the display screens shown in FIGS. 8 to 12. By knowing the degree of stress release, the subject can easily find a stress release method that suits them. In addition, the subject can know how much stress he/she will have for which event and can manage his/her own scheduling. Furthermore, by knowing the allowable remaining amount of stress, the subject can know how much stress there is no problem (whether there is any adverse effect on the mind and body). In addition, the person to be measured can grasp his/her stress state step by step by checking the message.

In the present embodiment, the stress release degree calculation apparatus 15 includes the stress release degree calculating unit 6, the long-term stress tolerable amount calculating unit 7, the long-term stress tolerable remaining amount, the stress degree calculating unit 8, the stress degree calculating unit 9, and the notifying unit 11. Although it is described as a thing, it is not necessary to have each of these parts. That is, the stress release degree calculation apparatus 15 may have at least a function of calculating the stress divergence degree based on the short-term stress value and the long-term stress value.

[Apparatus Operations]

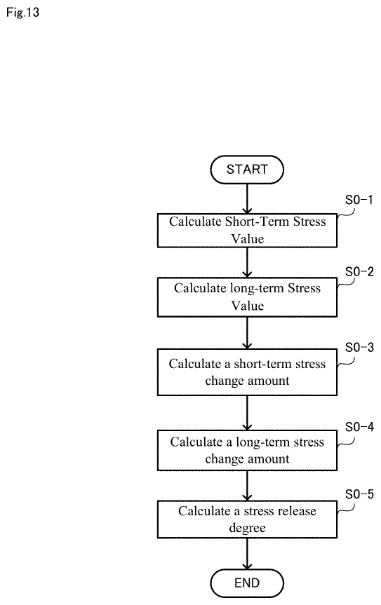

Next, the operations of the stress release degree calculation apparatus 15 will be described. FIGS. 13 to 18 are flow diagrams illustrating operations of the stress release degree calculation apparatus 15. In the present example embodiment, the stress management method is implemented by operating the stress release degree calculation apparatus 15. Therefore, the following description of the operations of the stress release degree calculation apparatus 15 applies to the stress management method according to the present example embodiment.

First, main operations of the stress release degree calculation apparatus 15 will be described with reference to FIG. 13. FIG. 13 is a flow diagram illustrating main operations of the stress release degree calculation apparatus 15.

The short-term stress calculating unit 1 calculates a short-term stress value, which indicates the amount of stresses felt by a subject in a first period (S0-1). The long-term stress calculating unit 2 calculates a long-term stress value, which indicates the amount of stresses felt by the subject in a second period (S0-2).

The short-term stress change amount calculating unit 3 calculates a short-term stress change amount of the subject (S0-3), based on the difference between short-term stress values calculated at different timings by executing the processing in step S0-1 at these timings. The long-term stress change amount calculating unit 4 calculates a long-term stress change amount of the subject (S0-4), based on the difference between long-term stress values calculated at different timings by executing the processing in step S0-2 at these timings.

The stress release degree calculating unit 5 calculates a stress release degree that indicates the extent of stress released by the subject based on the short-term stress change amount calculated in step S0-3 and the long-term stress change amount calculated in step S0-4 (S0-5).

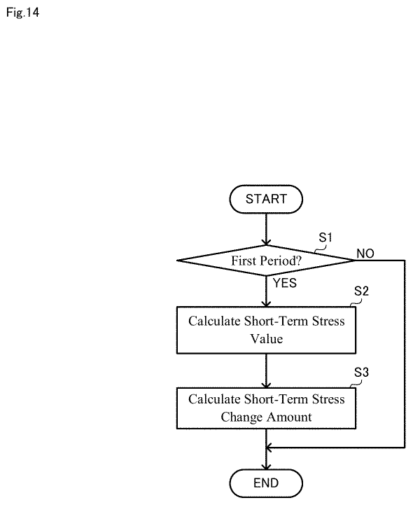

Hereinafter, specific operations of the stress release degree calculation apparatus 15 when calculating the short-term stress value, the long-term stress value, the short-term stress change amount, the long-term stress change amount, and the stress release degree will be described, with reference to FIGS. 14 to 16.

FIG. 14 is a flow diagram illustrating operations when calculating the short-term stress value, and the short-term stress change amount.

The short-term stress calculating unit 1 determines whether or not the first period has elapsed (S1). When step S1 is first executed, the short-term stress calculating unit 1 determines whether or not the first period has elapsed, after the stress management has started. Also, when step S1 is being performed for the second time or more, the short-term stress calculating unit 1 determines whether or not the first period has elapsed from the time when the short-term stress value was calculated immediately before.

Note that the short-term stress value can be calculated if first period-worth biological information has been obtained. Therefore, when the biological information is acquired regularly, even if a fixed period (first period) has not elapsed from when the short-term stress value was calculated immediately before, if first period-worth biological information has been acquired prior to a timing at which the short-term stress value is to be calculated, the short-term stress value is calculated.

If the first period has not elapsed (S1: NO), this flow is ended. If the first period has elapsed (S1: YES), the short-term stress calculating unit 1 calculates a short-term stress value using the method described above from the biological information acquired by the biological information acquiring unit 10 (S2). The biological information acquiring unit 10 may acquire the biological information at a timing at which the short-term stress value is to be calculated, or may also regularly acquire the biological information.

The short-term stress change amount calculating unit 3 calculates the short-term stress change amount using the method described above (S3). Here, if the short-term stress change amount cannot be calculated because the short-term stress value has only been calculated once, step S3 is not executed. Also, in step S3, the short-term stress change amount calculating unit 3 calculates a short-term stress release amount if the short-term stress value calculated in step S2 has decreased from the short-term stress value calculated immediately before, and calculates a short-term stress accumulation amount if the short-term stress value calculated in step S2 has increased.

Note that the short-term stress value and the short-term stress change amount are calculated in a series of processes in FIG. 14, but each process may also be executed in an independent flow.

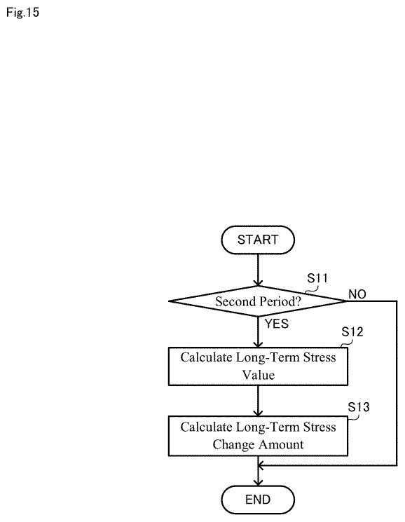

FIG. 15 is a flow diagram illustrating operations when calculating the long-term stress value, and the long-term stress change amount.

The long-term stress calculating unit 2 determines whether the second period has elapsed (S11). When step S11 is first executed, the long-term stress calculating unit 2 determines whether or not the second period has elapsed, after starting the stress management. Also, when the execution of step S11 is second time or thereafter, the long-term stress calculating unit 2 determines whether or not the second period has elapsed since the long-term stress value was calculated immediately before.

Note that the long-term stress value can be calculated if second period-worth biological information has been obtained. Therefore, when the biological information is acquired regularly, even if a fixed period (second period) has not elapsed since the long-term stress value was calculated immediately before, if second period-worth biological information has been acquired until a timing at which the long-term stress value is to be calculated, the long-term stress value is calculated.

If the second period has not elapsed (S11: NO), this flow is ended. If the second period has elapsed (S11: YES), the long-term stress calculating unit 2 calculates a long-term stress value (S12). The long-term stress calculating unit 2 calculates the long-term stress value from biological information acquired by the biological information acquiring unit 10 or the score of a questionnaire asked in advance, using the method described above. The score of a questionnaire may be calculated at a timing at which the long-term stress value is calculated, or may also be calculated when the result of a questionnaire is input to the stress release degree calculation apparatus 15.

The long-term stress change amount calculating unit 4 calculates the long-term stress change amount using the method described above (S13). Here, if the long-term stress release amount cannot be calculated because the long-term stress value is calculated only once, step S13 is not executed. Also, in step S13, the long-term stress change amount calculating unit 4 calculates a long-term stress release amount if the long-term stress value calculated in step S12 decreases from the long-term stress value calculated immediately before, and calculates a long-term stress accumulation amount if the long-term stress value calculated in step S12 increases.

Note that the long-term stress value and the long-term stress change amount are calculated in a series of processes in FIG. 15, but each process may also be executed in an independent flow.

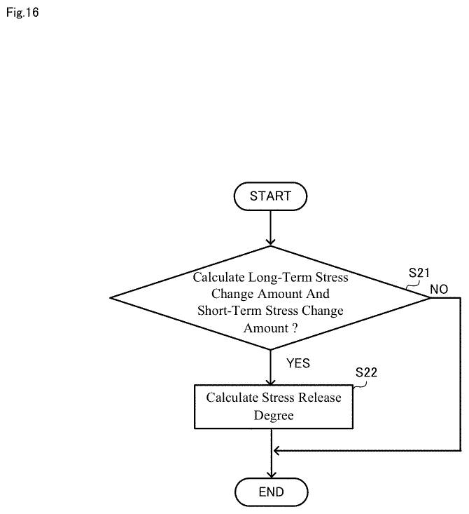

FIG. 16 is a flow diagram illustrating operations when the stress release degree is calculated.

The stress release degree calculating unit 5 determines whether the short-term stress change amount and the long-term stress change amount have been calculated (S21). If calculated (S21: YES), the stress release degree is calculated from the short-term stress change amount and the long-term stress change amount (S22), as described in FIG. 5. If not calculated (S21: NO), this flow is ended without calculating the stress release degree.

As described above, as a result of accurately calculating the stress release degree in a daily life, the stress release degree calculation apparatus 15 can cause a subject to recognize to what extent the stress is released. For example, as a result of notifying a subject of the calculated short-term stress value, long-term stress value, and stress release degree, as management information, the subject can recognize the state of stress felt by him/herself. The notification method includes screen output, as described in FIGS. 8, 9, and 12, audio output, or the like.

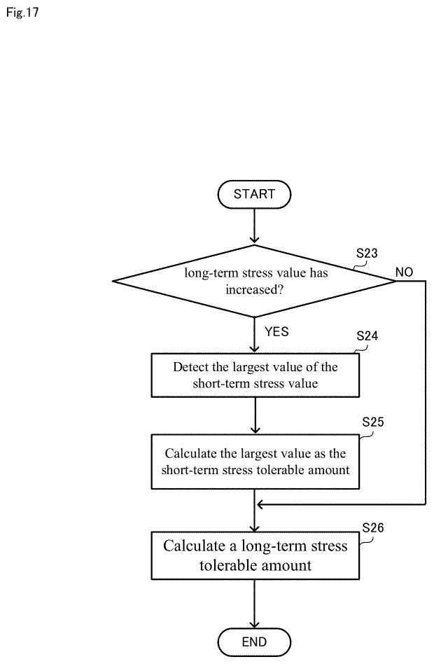

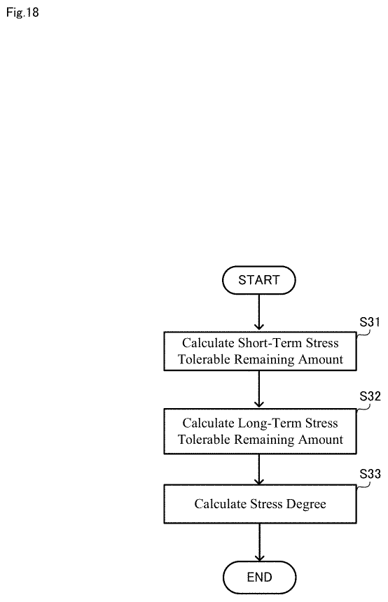

Next, the operations of the stress release degree calculation apparatus 15 when calculating the stress tolerable amount, the stress tolerable remaining amount, and the stress degree will further be described, with reference to FIGS. 17 and 18.

FIG. 17 is a flow diagram illustrating operations when calculating the stress tolerable amount.

The short-term stress tolerable amount calculating unit 6 determines whether the long-term stress value has increased (S23). For example, the short-term stress tolerable amount calculating unit 6 determines whether the increased amount of the long-term stress value from the value calculated at a certain timing to the value calculated immediately thereafter is less than a predetermined amount. If the long-term stress value has increased (S23: YES), the short-term stress tolerable amount calculating unit 6 detects the largest value of the short-term stress value in a period (period A in FIG. 6) in which the increased amount of the long-term stress value is less than the predetermined amount (S24), and calculates the largest value as the short-term stress tolerable amount (S25).

The long-term stress tolerable amount calculating unit 7 calculates a long-term stress tolerable amount (S26). For example, the long-term stress tolerable amount calculating unit 7 detects long-term stress values to be determined as a healthy state, and calculates the largest value, average value, or median value thereof as the long-term stress tolerable amount. After calculating the long-term stress tolerable amount, this flow is ended. If the long-term stress value has not increased (S23: NO), the long-term stress tolerable amount calculating unit 7 calculates the long-term stress tolerable amount (S26), because the long-term stress tolerable amount can be calculated, at a timing at which the long-term stress tolerable amount is calculated, by calculating the largest value or an average value of the long-term stress values in a previous certain period. Note that the processing for calculating the long-term stress tolerable amount in step S26 may be performed in a flow different from this processing.

FIG. 18 is a flow diagram illustrating operations when calculating the short-term stress tolerable remaining amount, the long-term stress tolerable remaining amount, and the stress degree. The stress release degree calculation apparatus 15 may regularly execute the flow shown in FIG. 18, or may also execute the flow when an operation made by a subject to start stress degree calculation is received.

The tolerable remaining amount calculating unit 9 calculates the short-term stress tolerable remaining amount (S31). Specifically, the tolerable remaining amount calculating unit 9 calculates the remaining amount of the short-term stress before the short-term stress tolerable amount is reached, that is, the difference between the current short-term stress value and the short-term stress tolerable amount, as the short-term stress tolerable remaining amount. Next, the tolerable remaining amount calculating unit 9 calculates the long-term stress tolerable remaining amount (S32). Specifically, the tolerable remaining amount calculating unit 9 calculates the remaining amount of the long-term stress before the long-term stress tolerable amount is reached, that is, the difference between the current long-term stress value and the long-term stress tolerable amount, as the long-term stress tolerable remaining amount.

The stress degree calculating unit 8 calculates the stress degree based on the short-term stress tolerable remaining amount calculated in step S31 and the long-term stress tolerable remaining amount calculated in step S32. The stress degree calculating unit 8 calculates the stress degree of stress felt by the subject from the correspondence table shown in FIG. 7, at a timing at which the long-term stress calculating unit 2 calculates the long-term stress value, for example.

Note that, in FIG. 18, the short-term stress tolerable remaining amount, the long-term stress tolerable remaining amount, and the stress degree are calculated in a series of processes, but each process may be performed in an independent flow.

The stress release degree calculation apparatus 15 further calculates the stress tolerable amount, the stress tolerable remaining amount, and the stress degree, and notifies a subject of these pieces of data as management information, and as a result, the subject can recognize the state of stress felt by him/herself in further detail. The notification method includes screen output, as described in FIGS. 10 to 12, and the like, audio output, or the like.

### [Program]

It is sufficient for the program according to the present example embodiment to be a program that causes a computer to execute each steps shown in FIGS. 13 to 18. The stress release degree calculation apparatus 15 and the stress management method according to the present example embodiment can be realized by installing this program in the computer and executing this program. In this case, the processor of the short-term stress calculating unit 1, the long-term stress calculating unit 2, the short-term stress change amount calculating unit 3, the long-term stress change amount calculating unit 4, the stress release degree calculating unit 5, the short-term stress tolerable amount calculating unit 6, a long-term stress tolerable amount calculating unit 7, a stress degree calculating unit 8, an tolerable remaining amount calculating unit 9, a biological information acquiring unit 10, and a notifying unit 11, and performs processing.

Further, the computer includes general-purpose PCs, smartphones and tablet-type terminal devices.

Also, the program in the present example embodiment may be executed by a computer system constructed by a plurality of computers. In this case, for example, each computer may function as one of the processor of the short-term stress calculating unit 1, the long-term stress calculating unit 2, the short-term stress change amount calculating unit 3, the long-term stress change amount calculating unit 4, the stress release degree calculating unit 5, the short-term stress tolerable amount calculating unit 6, a long-term stress tolerable amount calculating unit 7, a stress degree calculating unit 8, an tolerable remaining amount calculating unit 9, a biological information acquiring unit 10, and a notifying unit 11.

### [Physical Configuration of the Apparatus]

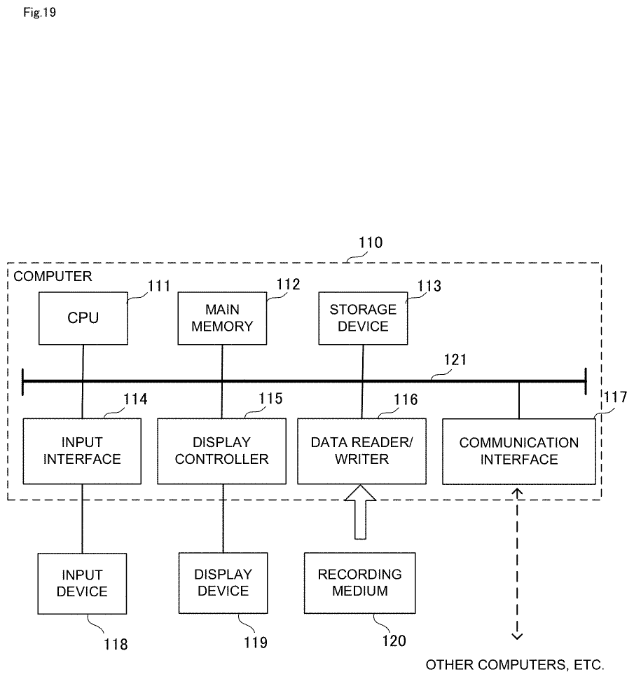

Hereinafter, a computer that realizes the stress release degree calculation apparatus 15 by executing the program in the example embodiment will be described with reference to FIG. 19. FIG. 19 is a block diagram showing one example of a computer that realizes the stress management apparatus 15.

As shown in FIG. 19, a computer 110 includes a CPU 111, a main memory 112, a storage device 113, an input interface 114, a display controller 115, a data reader/writer 116, and a communication interface 117. These components are connected in such a manner that they can perform data communication with one another via a bus 125. Note that the computer 110 may include a GPU (Graphics Processing Unit) or an FPGA (Field-Programmable Gate Array) in addition to the CPU 111 or in place of the CPU 111.

The CPU 111 carries out various types of computation by deploying the program (codes) in the present example embodiment stored in the storage device 113 to the main memory 112, and executing the deployed program in a predetermined order. The main memory 112 is typically a volatile storage device, such as a DRAM (Dynamic Random Access Memory). Also, the program in the present example embodiment is provided in a state where it is stored in a computer readable recording medium 120. Note that the program in the present example embodiment may also be distributed over the Internet connected via the communication interface 117.

Furthermore, specific examples of the storage device 113 include a hard disk drive, and also a semiconductor storage device, such as a flash memory. The input interface 114 mediates data transmission between the CPU 111 and an input apparatus 118, such as a keyboard and a mouse. The display controller 115 is connected to a display apparatus 119, and controls displays on the display apparatus 119. The data reader/writer 116 mediates data transmission between the CPU 111 and the recording medium 120, and executes readout of the program from the recording medium 120, as well as writing of the result of processing in the computer 110 to the recording medium 120. The communication interface 117 mediates data transmission between the CPU 111 and other computers.

Also, specific examples of the recording medium 120 include: a general-purpose semiconductor storage device, such as CF (Compact Flash®) and SD (Secure Digital); a magnetic recording medium, such as Flexible Disk; and an optical recording medium, such as CD-ROM (Compact Disk Read Only Memory).

A part or all of the aforementioned example embodiment can be described as, but is not limited to, the following (Supplementary Note 1) to (Supplementary Note 24).

(Supplementary Note 1)

A stress release degree calculation apparatus comprising:

a short-term stress calculation unit that calculates a short-term stress value that indicates stress felt by a subject in a first period;

a long-term stress calculation unit that calculates a long-term stress value that indicates stress felt by the subject in a second period that is longer than the first period and includes the first period;

a short-term stress change amount calculation unit that calculates a short-term stress change amount of the subject based on the difference between the short-term stress values calculated at different timings;

a long-term stress change amount calculation unit that calculates a long-term stress change amount of the subject based on the difference between the long-term stress values calculated at different timings; and

a stress release degree calculation unit that calculates a stress release degree indicating the extent of stress released by the subject based on the short-term stress change amount and the long-term stress change amount.

(Supplementary Note 2)

The stress release degree calculation apparatus according to Supplementary Note 1,

wherein the short-term stress change amount calculation unit,
if the short-term stress value change amount is a decreasing amount, calculates the short-term stress change amount as a short-term stress release amount, which is an amount of short-term stress released by the subject, and
if the short-term stress value change amount is an increasing amount, calculates the short-term stress change amount as a short-term stress accumulation amount, which is an amount of short-term stress accumulated by the subject, and

the long-term stress change amount calculation unit,
if the long-term stress value change amount is a decreasing amount, calculates the long-term stress change amount as a long-term stress release amount, which is an amount of long-term stress released by the subject, and
if the long-term stress value change amount is an increasing amount, calculates the long-term stress change amount as a long-term stress accumulation amount, which is an amount of long-term stress accumulated by the subject.

(Supplementary Note 3)

The stress release degree calculation apparatus according to Supplementary Note 2,

wherein the stress release degree increases as the long-term stress release amount increases relative to the short-term stress release amount.

(Supplementary Note 4)

The stress release degree calculation apparatus according to Supplementary Note 2 or 3,

wherein the stress release degree increases as the long-term stress release amount increases relative to the short-term stress accumulation amount.

(Supplementary Note 5)

The stress release degree calculation apparatus according to any one of Supplementary Notes 2 to 4,

wherein the stress release degree decreases as the long-term stress accumulation amount increases relative to the short-term stress release amount.

(Supplementary Note 6)

The stress release degree calculation apparatus according to any one of Supplementary Notes 2 to 5,

wherein the stress release degree decreases as the long-term stress accumulation amount increases relative to the short-term stress accumulation amount.

(Supplementary Note 7)

The stress release degree calculation apparatus according to any one of Supplementary Notes 1 to 6, further comprising

a biological information acquisition unit that acquires biological information regarding the subject,

wherein the short-term stress calculation unit calculates the short-term stress based on the biological information.

(Supplementary Note 8)

The stress release degree calculation apparatus according to Supplementary Note 7, wherein the biological information includes at least one of a perspiration amount, cutaneous temperature, body motion, a heart rate, an electrocardiogram, a pulse wave, a pulse, blood pressure, respiration, pupils, a brain wave, and myoelectric or gastric electrical information, of the subject.

(Supplementary Note 9)

A stress release degree calculation method comprising:

a step of calculating a short-term stress value that indicates stress felt by a subject in a first period;

a step of calculating a long-term stress value that indicates stress felt by the subject in a second period that is longer than the first period and includes the first period;

a step of calculating a short-term stress change amount of the subject based on the difference between the short-term stress values calculated at different timings;

a step of calculating a long-term stress change amount of the subject based on the difference between the long-term stress values calculated at different timings; and

a step of calculating a stress release degree indicating the extent of stress released by the subject based on the short-term stress change amount and the long-term stress change amount.

(Supplementary Note 10)

The stress release degree calculation method according to Supplementary Note 9,

wherein, in the step of calculating the short-term stress change amount,
if the short-term stress value change amount is a decreasing amount, the short-term stress change amount is calculated as a short-term stress release amount, which is an amount of short-term stress released by the subject, and
if the short-term stress value change amount is an increasing amount, the short-term stress change amount is calculated as a short-term stress accumulation amount, which is an amount of short-term stress accumulated by the subject, and

in the step of calculating the long-term stress change amount,
if the long-term stress value change amount is a decreasing amount, the long-term stress change amount is calculated as a long-term stress release amount, which is an amount of long-term stress released by the subject, and
if the long-term stress value change amount is an increasing amount, the long-term stress change amount is calculated as a long-term stress accumulation amount, which is an amount of long-term stress accumulated by the subject.

(Supplementary Note 11)

The stress release degree calculation method according to Supplementary Note 10,

wherein the stress release degree increases as the long-term stress release amount increases relative to the short-term stress release amount.

(Supplementary Note 12)

The stress release degree calculation method according to Supplementary Note 10 or 11,

wherein the stress release degree increases as the long-term stress release amount increases relative to the short-term stress accumulation amount.

(Supplementary Note 13)

The stress release degree calculation method according to any one of Supplementary Notes 10 to 12,

wherein the stress release degree decreases as the long-term stress accumulation amount increases relative to the short-term stress release amount.

(Supplementary Note 14)

The stress release degree calculation method according to any one of Supplementary Notes 10 to 13,

wherein the stress release degree decreases as the long-term stress accumulation amount increases relative to the short-term stress accumulation amount.

(Supplementary Note 15)

The stress release degree calculation method according to any one of Supplementary Notes 10 to 14, further comprising

a step of acquiring biological information regarding the subject,

wherein, in the step of calculating the short-term stress, the short-term stress is calculated based on the biological information.

(Supplementary Note 16)

The stress release degree calculation method according to Supplementary Note 15,

wherein the biological information includes at least one of a perspiration amount, cutaneous temperature, body motion, a heart rate, an electrocardiogram, a pulse wave, a pulse, blood pressure, respiration, pupils, a brain wave, and myoelectric or gastric electrical information, of the subject.

(Supplementary Note 17)

A computer-readable recording medium that includes a program including instructions recorded thereon, the instructions causing a computer to carry out:

a step of calculating a short-term stress value that indicates stress felt by a subject in a first period;

a step of calculating a long-term stress value that indicates stress felt by the subject in a second period that is longer than the first period and includes the first period;

a step of calculating a short-term stress change amount of the subject based on the difference between the short-term stress values calculated at different timings;

a step of calculating a long-term stress change amount of the subject based on the difference between the long-term stress values calculated at different timings; and

a step of calculating a stress release degree indicating the extent of stress released by the subject based on the short-term stress change amount and the long-term stress change amount.

(Supplementary Note 18)

The computer readable recording medium according to Supplementary Note 17,

wherein, when the short-term stress change amount is caused to be calculated,
if the short-term stress value change amount is a decreasing amount, the short-term stress change amount is calculated as a short-term stress release amount, which is an amount of short-term stress released by the subject, and
if the short-term stress value change amount is an increasing amount, the short-term stress change amount is calculated as a short-term stress accumulation amount, which is an amount of short-term stress accumulated by the subject, and

when the long-term stress change amount is caused to be calculated,
if the long-term stress value change amount is a decreasing amount, the long-term stress change amount is calculated as a long-term stress release amount, which is an amount of long-term stress released by the subject, and
if the long-term stress value change amount is an increasing amount, the long-term stress change amount is calculated as a long-term stress accumulation amount, which is an amount of long-term stress accumulated by the subject.

(Supplementary Note 19)

The computer readable recording medium according to Supplementary Note 18,

wherein the stress release degree increases as the long-term stress release amount increases relative to the short-term stress release amount.

(Supplementary Note 20)

The computer readable recording medium according to Supplementary Note 18 or 19,

wherein the stress release degree increases as the long-term stress release amount increases relative to the short-term stress accumulation amount.

(Supplementary Note 21)

The computer readable recording medium according to any one of Supplementary Notes 18 to 20,

wherein the stress release degree decreases as the long-term stress accumulation amount increases relative to the short-term stress release amount.

(Supplementary Note 22)

The computer readable recording medium according to any one of Supplementary Notes 18 to 21,

wherein the stress release degree decreases as the long-term stress accumulation amount increases relative to the short-term stress accumulation amount.

(Supplementary Note 23)

The computer readable recording medium according to any one of Supplementary Notes 18 to 22, the program further including instructions that causes the computer to carry out

a step of acquiring biological information of the subject,

wherein, when the short-term stress is caused to be calculated, the short-term stress is calculated based on the biological information.

(Supplementary Note 24)

The computer readable recording medium according to Supplementary Note 23,

wherein the biological information includes at least one of a perspiration amount, cutaneous temperature, body motion, a heart rate, an electrocardiogram, a pulse wave, a pulse, blood pressure, respiration, pupils, a brain wave, and myoelectric or gastric electrical information, of the subject.

While the present invention has been described above with reference to the example embodiment, the present invention is not limited to the aforementioned example embodiment. Various changes that can be understood by a person skilled in the art within the scope of the present invention can be made to the configurations and details of the present invention.

### REFERENCE SIGNS LIST

1 short-term stress calculating unit
2 long-term stress calculating unit
3 short-term stress change amount calculating unit
4 long-term stress change amount calculating unit
5 stress release degree calculating unit
6 short-term stress tolerable amount calculating unit
7 long-term stress tolerable amount calculating unit
8 stress degree calculating unit
9 tolerable remaining amount calculating unit
10 biological information acquiring unit
11 notifying unit
15 stress release degree calculation apparatus
20 wearable terminal
21 display device
110 computer
111 CPU
112 main memory
113 storage device
114 input interface
115 display controller
116 data reader/writer
117 communication interface
118 input apparatus
119 display apparatus
120 recording medium
121 bus

What is claimed is:

1. A stress release degree calculation apparatus comprising:
a short-term stress calculation unit that calculates a short-term stress value that indicates stress felt by a subject in a first period;
a long-term stress calculation unit that calculates a long-term stress value that indicates stress felt by the subject in a second period that is longer than the first period and includes the first period;
a short-term stress change amount calculation unit that calculates a short-term stress change amount of the subject based on the difference between the short-term stress values calculated at different timings;
a long-term stress change amount calculation unit that calculates a long-term stress change amount of the subject based on the difference between the long-term stress values calculated at different timings; and
a stress release degree calculation unit that calculates a stress release degree indicating the extent of stress released by the subject based on the short-term stress change amount and the long-term stress change amount.

2. The stress release degree calculation apparatus according to claim 1,
wherein the short-term stress change amount calculation unit,
if the short-term stress value change amount is a decreasing amount, calculates the short-term stress change amount as a short-term stress release amount, which is an amount of short-term stress released by the subject, and
if the short-term stress value change amount is an increasing amount, calculates the short-term stress change amount as a short-term stress accumulation amount, which is an amount of short-term stress accumulated by the subject, and
the long-term stress change amount calculation unit,
if the long-term stress value change amount is a decreasing amount, calculates the long-term stress change amount as a long-term stress release amount, which is an amount of long-term stress released by the subject, and
if the long-term stress value change amount is an increasing amount, calculates the long-term stress change amount as a long-term stress accumulation amount, which is an amount of long-term stress accumulated by the subject.

3. The stress release degree calculation apparatus according to claim 2,
wherein the stress release degree increases as the long-term stress release amount increases relative to the short-term stress release amount.

4. The stress release degree calculation apparatus according to claim 2,
wherein the stress release degree increases as the long-term stress release amount increases relative to the short-term stress accumulation amount.

5. The stress release degree calculation apparatus according to claim 2,
wherein the stress release degree decreases as the long-term stress accumulation amount increases relative to the short-term stress release amount.

6. The stress release degree calculation apparatus according to claim 2,
wherein the stress release degree decreases as the long-term stress accumulation amount increases relative to the short-term stress accumulation amount.

7. The stress release degree calculation apparatus according to claim 1, further comprising
a biological information acquisition unit that acquires biological information regarding the subject,
wherein the short-term stress calculation unit calculates the short-term stress based on the biological information.

8. The stress release degree calculation apparatus according to claim 7,
wherein the biological information includes at least one of a perspiration amount, cutaneous temperature, body motion, a heart rate, an electrocardiogram, a pulse wave, a pulse, blood pressure, respiration, pupils, a brain wave, and myoelectric or gastric electrical information, of the subject.

9. A stress release degree calculation method comprising:
calculating a short-term stress value that indicates stress felt by a subject in a first period;
calculating a long-term stress value that indicates stress felt by the subject in a second period that is longer than the first period and includes the first period;
calculating a short-term stress change amount of the subject based on the difference between the short-term stress values calculated at different timings;
calculating a long-term stress change amount of the subject based on the difference between the long-term stress values calculated at different timings; and
calculating a stress release degree indicating the extent of stress released by the subject based on the short-term stress change amount and the long-term stress change amount.

10-16. (canceled)

17. A non-transitory computer-readable recording medium that includes a program including instructions recorded thereon, the instructions causing a computer to carry out:
calculating a short-term stress value that indicates stress felt by a subject in a first period;
calculating a long-term stress value that indicates stress felt by the subject in a second period that is longer than the first period and includes the first period;
calculating a short-term stress change amount of the subject based on the difference between the short-term stress values calculated at different timings;
calculating a long-term stress change amount of the subject based on the difference between the long-term stress values calculated at different timings; and
calculating a stress release degree indicating the extent of stress released by the subject based on the short-term stress change amount and the long-term stress change amount.

18-24. (canceled)
